(19) United States
(12) Patent Application Publication
(10) Pub. No.: US 2006/0067896 A1
(43) Pub. Date: Mar. 30, 2006

(54) Composition of sunscreen and color-changing markers

(76) Inventor: Robin Schaffer, Englewood, CO (US)

Correspondence Address: LATHROP & GAGE LC, 4845 PEARL EAST CIRCLE, SUITE 300, BOULDER, CO, 80301, US

(21) Appl. No.: 11/240,876

(22) Filed: Sep. 30, 2005

Related U.S. Application Data
(60) Provisional application No. 60/614,959, filed on Sep. 30, 2004.

(51) Int. Cl. A61K 8/49; A61K 8/37

(52) U.S. Cl. 424/59

(57) ABSTRACT

A sunscreen/sunblock product that combines sun protection and color marking activities intended to encourage use of sunscreen, especially among young children. The sunscreen/sunblock product also enables a user to visualize when the product needs to be re-applied. The sunscreen composition incorporates a color-triggering developer into commercially available sunscreens. This sunscreen product may be used as a regular colorless or colored sunscreen. A marking composition containing a color precursor is also prepared. A marking device may be utilized to store and/or apply the marking composition to the skin which is covered with a thin layer of the sunscreen product. Color markings are formed through the reaction of the color-triggering developer and the color precursor. The color markings typically stay on the skin only as long as the sunscreen is on the skin, thus providing an indication that more sunscreen needs to be applied for adequate sun protection. The color markings are also washable in the event that the color is inadvertently transferred to an unintended surface.

### RELATED APPLICATIONS

This application claims benefit of priority to U.S. provisional application Ser. No. 60/614,959 filed Sep. 30, 2004, the disclosure of which is herein incorporated by reference.

### BACKGROUND

1. Field of the Invention

The present invention pertains generally to skin care products, such as lotions, sunscreens, sunblocks, and other skin-protection products and cosmetics. More particularly, the system and composition described here relate to sunscreen products that are designed to increase wearing of sunscreen, or alternatively as a reminder to re-apply sunscreen. It is recognized that the system and compositions disclosed may also be used in other skin care products and cosmetics.

2. Description of the Related Art

It is widely recognized that sunscreen is useful in the reduction of skin damage and certain types of skin cancers. See Saraiyam, M. et al., Interventions to Prevent Skin Cancer by Reducing Exposure to Ultraviolet Radiation—A Systematic Review, Am. J. Preventive Med., 27:422-66, at 424 (2004). Despite extensive public awareness programs aiming to promote the use of sunscreen, many studies have suggested that children continue to underutilize sunscreens or sun-protective clothing. See e.g., Grin C. M. et al., Sun Exposure of Young Children while at Day Care. Pediatr Dermatol. 11:304-309 (1994).

Sunscreen companies have marketed colored sunscreens in an effort to encourage use of sunscreen among children. Even so, the inherent messiness of these products is a significant deterrent to widespread use. Many of the colored sunscreens are zinc based, and this means that they tend to remain wet for a time after being applied to human skin. If the wet sunscreen contacts an object, such as a shirt or a towel, the color of the sunscreen may transfer as an unsightly stain. Accordingly, there exists a need for a sunscreen product that avoids the formation of inadvertent stains on household objects or other unintended surfaces.

A certain class of what may be characterized as invisible inks operate to produce color when a color precursor reacts with a developer. In one example of this, U.S. Pat. No. 6,124,377 issued to Kaiser et al. describes a marker system that is used as an aid to drawing or writing. A paper or plastic substrate contains a color-triggering developer that undergoes a chemical reaction to produce color upon the application of a color precursor. U.S. Pat. No. 6,124,377 is hereby incorporated by reference to the same extent as though fully disclosed herein.

### SUMMARY

The present disclosure addresses a sunscreen system that overcomes the problems outlined above and advances the art by providing a sunscreen that is formulated to contain a color-triggering developer which is colorized by the application of a color precursor. Once the sunscreen is applied to the skin, the color precursor may be applied by a marking device, such as a roller, pen, marker or wipe. Alternatively, a second lotion that contains the color precursor may be applied by hand to create, for example, handprints or finger-paint designs. Color markings may thus be created at selected locations on the skin. The sunscreen product disclosed herein is fun to wear and less messy to the general environment.

The terms “color-triggering developer” and “color precursor” are used generally herein to describe a system of complementary reagents that react with one another to produce a color. For simplicity and consistency of terminology, the term “color precursor” as used herein refers to one of these complimentary reagents that is used as a marking composition, i.e., the color precursor is applied to the sunscreen and the sunscreen contains the color-triggering developer. By way of example, one embodiment uses a leuco dye or leuco dye intermediate as the color precursor in a marking composition. The color-triggering developer in the sunscreen is a Lewis acid, such as an activated clay, phenolic resin, zinc containing resin, and combinations thereof. Alternatively and unless otherwise specifically noted herein, the precursor and developer materials may be switched in their complementary roles, such that the color-triggering developer in the sunscreen is the activated clay, phenolic resin, zinc containing resin, and the color precursor in the marking composition is the leuco dye.

In one embodiment, the marking device is a marker pen or roller-dispenser that dispenses a formulation of ingredients including the color precursor. In one aspect, the ability to create these designs encourages young children to use sunscreen because the children desire to wear the designs. In another aspect, adults may monitor the need to reapply sunscreen as the color marking fade.

Since the color-triggering developer is mixed with the sunscreen and this is top-coated with the color precursor, the color markings disappear or fade as the sunscreen wears off. It is time to reapply more sunscreen when the color markings fade or disappear. Thus, the present disclosure provides a useful reminder for the sunscreen wearers and their care-takers to reapply the sunscreen.

In one embodiment, the color-triggering developer is colorless before the color precursor is applied. Accidental contact by the wearer with other surfaces does not cause color transfer as observed in prior colored sunscreen products. Even after color markings have been developed on the skin, accidental staining on an unintended surface is usually washable because the color markings are water soluble. Moreover, the color markings and all materials used according to this disclosure are non-toxic and are generally regarded as safe for small children.

The color developing ingredients used herein are generally nonreactive or stable when combined with the active UV-blocking ingredients in the sunscreen. The sunscreen products according to the present disclosure therefore are not compromised for their capability to protect wearers from UV exposure and skin damage.

Typically, a sunscreen lotion that contains the color triggering developer is applied to bare human skin. The color precursor to be applied by the marking device may include at least one leuco dye intermediate. Writing or stamping with the marking device on the sunscreen-coated skin facilitates a chemical reaction that produces color on the skin corresponding to the selected color of the leuco dye intermediate. Little or no coloration occurs by using the marking device on surfaces other than the part of the skin that is covered with the sunscreen.

The marking instrument can be in any of a number of different forms, including a marker, a pencil, a pen, a crayon, a stamp, a brush, or even a human body part such as a hand or finger. One or more additives, such as, for example, surfactants, antioxidants, buffers, dye blockers, and/or halogen-containing flame retardants can be included in the marking developer composition.

### BRIEF DESCRIPTION OF THE DRAWINGS

FIG. 1 shows a sunscreen formulation that has been applied to human skin as a color precursor is being applied to provide a color reaction on a selected portion of the human skin.

### DETAILED DESCRIPTION

In accordance with the present disclosure, the composition to be applied through a marking device includes at least one color precursor, particularly, a leuco dye intermediate. The leuco dye remains colorless until reacted with a color-triggering developer.

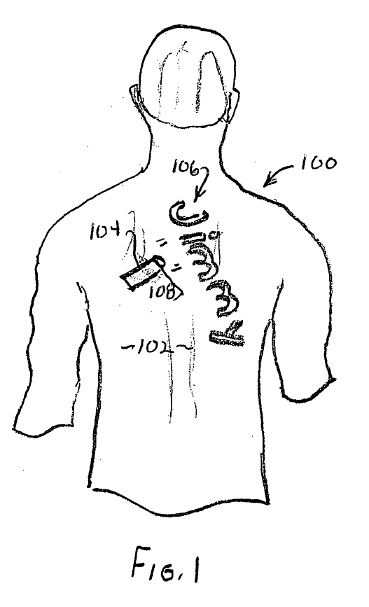

As shown in FIG. 1, an individual 100 presents human skin as a bare back 102 to which a sunscreen formulation has been applied. The sunscreen formulation is not shown in FIG. 1 because the sunscreen formulation is clear, but the sunscreen formulation covers the entire area of back 102. An applicator has been manually manipulated to write indicia n a selected portion of back 102. As shown, the applicator is a roller device having a ball 106 that is coated with the liquid contents of applicator 104 as the ball rotates when in contact with human skin. This rotating action dispenses the contents over a path of travel shown as indicia 106. The indicia 106 is formed as a chemical reaction between the sunscreen formulation and the contents of applicator 104. Alternatively, the applicator may be a stamp or other type of dispenser. By way of example, the indicia 106 may be a design, a cartoon character, a washable tattoo, a time of application, a name, a slogan, or a mark. The indicia 106 may fade in intensity as the individual 100 engages in activities under exposure to the sun. Visibly perceived fading is an indicator that the sunscreen formulation needs to be reapplied.

The marking composition is applied by use of a marking device to human skin on which sunscreen base has been applied. The marking device may be a marker, pencil, crayon, stamp, stamp pad, pen, brush, roller, wipe, or and other dispensing device that contains the color precursor made ready for dispensation. Body parts, such as a human finger, hand or foot, can be used to apply the marking composition. In addition, the marking composition can be in the form of paint, and can therefore be applied by way of a paintbrush and sprayers. The marking composition also desirably contains one or more compatible ingredients, for example, surfactants, antioxidants, buffers (such as, for example, non-water-soluble tertiary amines), dye blockers, and/or halogen-containing flame retardants. The additives may be provided in any effective amount.

The color precursor preferably contains at least one leuco dye intermediate. The leuco dye intermediates are sensitive to oxidation and/or acidic environments. Examples of suitable leuco dyes include, but are not limited to, diarylphthalide dyes, fluoran dyes, indolyphthalide dyes, acylluecoazine dyes, leucoauramine dyes, spiropyrane dyes, rhodaminelactam dyes, triarylmethane dyes and chromene dyes. Preferred leuco dyes include, but are not limited to, Spiro(12H-benzo(a)xanthene-12,1′(3′H)-isobenzofuran-3′-one,9-(diethylamino ) (such as COPIKEM.RTM. 747), 3-[Butyl-2-methylindol-3-yl]-3-(1-octyl-2-methylindol-3-yl)-1(3H)isobenzofuranone (such as COPIKEM.RTM. 35 magenta), 2-′phenylamino-3′-methyl-6′-(dibutylamino) spiro[isobenzofuran-1(3H),9′-(9H)-xanthen]-3-one (such as COPIKEM.RTM. 34 Black), substituted Phthalide (such as COPIKEM.RTM. 14 Orange), such as COPIKEM.RTM. 7 Grape, 2′Di(phenylmethyl)amino-6′(diethylamino)spiro(isobenzofuran-1(3H),9′-(9H)xanthen)-3-one (such as COPIKEM.RTM. 5 green). Products identified under the COPIKEM.RTM. trademarks are commercially available from the Hilton Davis Company, Cincinnati, Ohio.

The leuco dye intermediates may be present in a concentration suitably from about 0.5% to about 6% by weight of the marking composition. For example, the leuco dye intermediates may be present in a concentration of about 0.5% to about 2% by weight of the composition. However, some leuco dye intermediates may be provided, especially in combination, in higher concentrations. By way of example, as is known in the art a black chromophore may be provided by utilizing a combination of three different black leuco dyes, each provided in a concentration of about 2% by weight of the composition.

The leuco dye intermediate is desirably introduced into a liquid medium, which is used as a carrier or vehicle. Preferably, the color precursor is dissolved in solvent. The solvent (or combination of solvents) is desirably substantially optically clear and is selected such that the solubilized dye gives good color formation when reacted with the color-triggering developer (i.e., the solvent should solubilize a sufficient concentration of the leuco dye such that good color formation, based upon visual observation, ultimately results after color-triggering occurs). The solvent is preferably miscible with that of the sunscreen formulation.

It is preferred that the leuco dye should not precipitate out of solution at room temperature over time. The propensity of the dye to precipitate from a particular formulation may be tested by utilizing freeze-thaw cycles. For example, in one such test the dyes should not precipitate out of solution when exposed to about 4-5 freeze-thaw cycles. In addition, the solvent desirably has a dynamic viscosity of about 12 centipoise or less (and more preferably 10 centipoises or less), in order to be suitable for application in a capillary system, as is particularly desirable for the present purposes.

Preferred solvents are non-toxic and non-flammable (e.g., flash points above about 280° F.), and exhibit low volatility. In addition, the solvents are substantially non-odorous. Strictly by way of example, the solvent for carrying the color precursors may be selected from, for example, dimethyl adipate, diethyl succinate, dibutyl phthalate, chlorinated and fluorinated toluenes, such as parachlorobenzotrifluoride (e.g., OXSOL.RTM. 100, commercially available from Occidental Chemical Corporation, Dallas, Tex.), or combinations thereof. Although OXSOL.RTM. 100 is an otherwise desirable solvent in many respects, its mothball-like odor makes it less desirable. It is worth noting that odor masks may be utilized to attenuate odors in some solvents. The solvent is preferably present in a concentration ranging from about 30% to about 90% by weight of the marking composition, and is more preferably in a concentration ranging from about 55% to about 75% by weight of the composition.

The marking composition may also include at least one buffer, such as an amine mentioned above, in order to inhibit the premature acidification of the dyes. In this regard, some solvents contain acid functionalities that may trigger the formation of chromophores exhibiting the desired color, but prematurely. In response, amines provide a versatile means of buffering the solution of the composition. The most desirable buffers are non-volatile amines that do not evaporate. It is important that the buffers exhibit little or no water solubility and have a relatively high boiling point (e.g., 100° C. or higher). In this regard, minimizing the water solubility is important because the buffer might otherwise “wash out,” for example, when exposed to detergents in a washing machine. Since detergents often contain acid moieties, the color could actually develop on a fabric while in the washer. For example, if the molecular weight of the buffer is too low, the buffer can emulsify and undesirably wash out. In addition, if the boiling point is too low, the buffers can be removed via evaporation in clothes dryers, which commonly operate at temperatures of about 80-85° C.

The amine may be suitably effective in a concentration as low as about 0.0002% by weight of the marking composition. Preferably, the amine is present in a concentration of about 0.05% to about 1.50% by weight of the composition. The amines may be selected from, for example, tributyl amine, tridodecyl amine, dimethylpiperdone (DMPD), dicyclohexyl amine (DCHA), 4,4′-methylenebiscyclo hexanamine, diazabycyclo (5.4.0) undec-7-ene-1,8, mixed polycyclo aliphatic amine, tetramethyl propylenediamine, 1,3-diaminopropane, 1,7-diaminoheptane,1,8-diaminooctane, 1,9-diaminodelane, 1,12-diaminododecane, octadecylamine, diethylcyclohexylamine, Jeffamine D-230, Jeffamine XTJ-502, Jeffamine XTJ-504, Jeffamine XTJ-510, Jeffamine T-5000, tributyl amine, trioctylamine or combinations thereof It is to be noted that Jeffamine products are commercially available from Huntsman. While primary, secondary, tertiary, and quaternary amines may be used, tertiary amines such as, for example, tributyl amine, trihexyl amine, trioctyl amine, tridecyl amine, tridodecyl amine, and combinations thereof work best and generally exhibit the lowest order of toxicity.

The marking composition is preferably provided with at least one antioxidant in order to prevent the premature oxidation of the dyes. Inasmuch as the dyes are bathed in an atmosphere of oxygen, eventually they can oxidize (without intervention) once they are in solution, such as during storage in a marker or other marking instrument. The antioxidants are therefore provided to prevent premature chromophore development. Examples of suitable antioxidants include, but are not limited to, Vitamin E, Tinuvins (commercially available from Ciba Geigy), 3,5-bis (1,1-dimethylethyl)-4-hydroxy benzenepropanoic acid,2,2-bis[[3-[3.5-bis(1,1-dimethylethyl)-4-hydroxyphenyl]-1-oxopropoxy]methyl]1,3-propanediyl ester (commercially available from Ciba as Irganox 1010), Irganox 1076 (commercially available from Ciba), butylated hydroxytoluene (BHT), butylated hydroxyanisole (BHA), e.g., tert-butylhydroquinone (TBHQ), and combinations thereof. Desirably, the antioxidants are substantially non-soluble to water (so as to avoid washing out) and are not volatile when exposed to heat. With respect to volatilization, the antioxidants should desirably resist volatilization and evaporation at temperatures typically found in operating clothes dryers (such as temperatures of about of 80-85° C.). The amount of antioxidant provided in the marking composition can vary depending on the chemistry of the particular antioxidant. For example, Vitamin E is suitable at levels of about 5% by weight of the marking composition or higher. Other antioxidants, such as the Tinuvins, work well at lower concentrations, such as at about 2% by weight of the marking composition. It is to be appreciated that ultraviolet absorbing materials which are normally present in the sunscreen base may help inhibit the fading of the developed colors. Tinuvins are helpful in preventing color fading as well.

Surfactants in any effective amount may be a useful ingredient of the marking compositions. Examples of suitable surfactants include, for example, acetylinic diol-based compounds, such as, for example, tetramethyl decynediol in a 2-ethyl hexanol solvent (such as Surfynol 104A, commercially available from Air Products, Inc.), ethoxylated acetylenic diols (such as Dynol 604, commercially available from Air Products, Inc.), Witconate P-1059, Witcoamide 511, Witcoamide 5138, Surfynol CT-171, Surfynol CT-111, Surfynol CT-131, Surfynol TG, DBE Microemulsion, Fluorad FC-431, Fluorad FC-430, Surfynol 104A, Dynol 604 and combinations thereof. It is to be noted that Witco products are commercially available from Witco Corporation and that Fluorad products are commercially available from 3M. Preferably, the surfactants are provided in a concentration of about 1% to about 30% by weight of the marking composition. The surfactants are provided in order to modify properties such as viscosity and surface tension of the compositions in order to improve dye solubility and act as an anti-redeposition agent of the dye on fabrics when washed. Preferably, the surfactant, or surfactants as combined, have an hydrophilic-lipophilic balance score of about 6 or less, and more preferably from about 3 to about 6. Hydrophilic-lipophilic balance (HLB) identifies the relative hydrophobicity and hydrophilicity of a surfactant and ranges from 0 to 14. Surfactants having HLB scores of about 3 to about 6 are most desired because those surfactants are more hydrophobic than hydrophilic, and hence, more soluble in solvents than in water, but still are partially soluble in water. Advantageously, the desired HLB rating may be achieved even when mixing surfactants. For example, Surtynol TG has an HLB that is too high for use alone. However, Surfynol TG may be combined with a surfactant having a much lower HLB so that the combined surfactants can have the most desired HLB falling within the range of about 3 to 6. As such, preferable surfactants permit enhanced dye solubility, yet still permit washing from fabric so as to avoid staining of the fabric.

With respect to water, a dibasic ester or other microemulsion concentrate (such as the dibasic ester microemulsion concentrate available from DuPont) may be utilized to enhance the inclusion of water. In this regard, water is typically not desired in the system because of the potential for premature color development. It is believed that water can act like a Lewis acid and trigger inadvertent color development. However, some solvents used for solubilizing the dye in the marking composition can “attack” (i.e., dissolve the developer or developed color markings) without intervention. As a result, developed color can be transferred undesirably to clothing or household surfaces. When employing such developer-attacking solvents, the inclusion of water, for example, by way of a microemulsion concentrate, beneficially provides a “barrier” to this undesirable transfer of developed color. Particularly, the water precludes the solvent from reacting with the color-developing composition until the water flashes off (e.g., after just a second or few seconds). This delay sufficiently prevents the color transfer or “pick-up”. Further, in order to prevent premature coloration due to the presence of water, the water may be desirably pre-neutralized with a buffer, such as a water-soluble amine.

Flame retardants of conventional types (such as, for example, halogen containing flame retardants) can be optionally employed in the marking compositions. Examples of suitable flame retardants include trisnonylphonyl phosphite (e.g., DOVERPHOS 4.RTM., commercially available from Dover Chemical Corporation, Dover, Ohio), a liquid brominated, chlorinated olefin (e.g., DOVERGUARD 8426, commercially available from Dover Chemical Corporation, Dover, Ohio), and combinations thereof. The flame retardants are particularly desirable because they prevent ignition, particularly of associated materials, such as plastic found in the marker or other marking instrument. Flame retardants are more useful in marking compositions that employ solvents having flash points below about 280° F. (about 137° C.). Furthermore, it has been found that halogen containing flame retardants can often provide the added benefit of serving as dye solubilizers. Accordingly, the concentration of the flame retardant is preferably in a range of about 25% to about 100% by weight of the solvent in the marking composition.

In addition, the marking composition can also desirably include at least one dye blocker, such as, for example, a novolac epoxy, tannic acid, and any combination thereof. The dye blocker is desirably soluble in organic solvents such as an aforementioned suitable solvent selected for inclusion in the marking composition. Advantageously, the dye blockers prevent or inhibit color development on fabrics, especially when the fabric is exposed to high temperatures, as can be typically encountered in washing machines operated at high temperatures and clothes dryers operated at high temperatures. Preferably, the dye blocker is provided in a concentration of about 0.5% by weight of the marking composition to about 15% by weight of the marking composition. Most preferably, the dye blocker is provided in a concentration of about 0.5% to about 3% by weight of the marking composition.

The sunscreen base may contain a commercially available sunscreen products. These sunscreen products may contain a sunscreening effective amount of one or more oil-soluble or water-soluble sunscreening UV-B actives or a mixture of one or more UV-B actives and one or more UV-A actives. UV-A type sunscreening actives protect against long wavelength actinic radiation of the sun in the 320 to 400 nm range while UV-B type sunscreening actives protect against shorter wavelength, actinic radiation of the sun in the 290-320 nm range. A sunscreen formulation is prepared by mixing the sunscreen base with an effective amount of a color-triggering developer.

The present composition may desirably contain opacifying agents, such as zinc oxide or titanium dioxide, which function by providing an opaque barrier to UV light. The present composition may desirably contain colored substances which attenuate light, such as red petrolatum. The preferred sunscreen products contain one or ingredients that function by selectively absorbing ultraviolet light having wavelengths which are responsible for sun related skin damage. Specific examples of these ingredients and their concentrations ranges are known to the art in approved concentrations. Examples of such ingredients include, for example, octocrylene; 2-ethylhexyl salicylate; 2-ethoxyethyl-p-methoxycinnamate; 2-ethylhexyl-p-methoxycinnamate (also known as octyl methoxycinnamate); 2-[bis(2-hydroxyethyl)-amino]ethyl salicylate; hydroxyethylaminoethyl-p-methoxycinnamate; pentyl 4-dimethylaminobenzoate; octyl dimethyl p-aminobenzoic acid; pentyl dimethyl p-aminobenzoic acid; ethyl dihydroxypropyl p-aminobenzoic acid; 2-hydroxy-1,4-naphthoquinone; 3,3,5-trimethylcyclohexyl salicylate; 2-ethylhexyl-2-cyano-3,3-diphenyl acrylate; ethyl 4-[bis(2-hydroxypropyl)amino]benzoate; digalloyl trioleate; menthyl anthranilate; salicylic acid 3,3,5-trimethyl ester (commonly known as homomenthyl salicylate or homosalate); glyceryl-p-aminobenzoate; p-aminobenzoic acid; isobutyl-p-aminobenzoate; isoamyl-p-dimethylaminobenzoate; 2-hydroxy-4-methoxybenzophenone-5-sulfonic acid; 2-phenylbenzimidazole-5-sulfonic acid; 2-2-dihydroxy-4-methoxybenzophenone; 2-hydroxy-4-methoxybenzophenone (commonly called oxybenzone); butylmethoxydibenzoylmethane (also known as avobenzone and sold under the trade name PARSOL 1789); benzophenone-8; benzophenone-4; benzophenone-3; 4-mono(3-hydroxypropyl)amino isomer of ethyl benzoate; 4-bis(3-hydroxypropyl)amino isomer of ethyl benzoate; 2-ethylhexyl-p-dimethylaminobenzoate; sulfomethyl benzylidene bornanone; urocanic acid and its esters; and mixtures thereof.

Typical sunscreen actives include para-aminobenzoic acid up to about 15 weight percent or from about 5 to 15% in admixture with other sunscreen actives; cinoxate up to about 3 weight percent or about 1 to 3% in admixture; diethanolamine methoxycinnamate up to 10 weight percent or about 8 to 10% in admixture; digalloyl trioleate up to 5 weight percent or about 2 to 5% in admixture; dioxybenzone up to 3 weight percent alone or in admixture; ethyl 4-[bis(hydroxypropyl)]aminobenzoate up to 5 weight percent or about 1 to 5% in admixture; glyceryl aminobenzoate up to 3 weight percent or about 2 to 3% in admixture; homosalate up to 15 weight percent or about 4 to 15% in admixture; lawsone up to 0.25 weight percent with dihydroxyacetone up to 3 weight percent, or in admixture 0.25 weight percent lawsone and 3 wt. % dihydroxyacetone; menthyl anthranilate up to 5 weight percent or about 3.5 to 5% in admixture; octocrylene up to 10 weight percent or 7 to about 10% in admixture; octyl methoxycinnamate up to 7.5 weight percent or about 2 to 7.5% in admixture; octyl salicylate or 2-ethylhexyl salicylate up to 5 weight percent or about 3 to 5% in admixture; oxybenzone up to 6 weight percent or about 2 to 6% in admixture; padimate “O” up to 8 weight percent or about 1.4 to 8% in admixture; phenylbenzimidazole sulfonic acid up to 4 weight percent or about 1 to about 4% in admixture; red veterinary petrolatum up to 95 percent or about 30 to 95% in admixture; sulisobenzone up to 10 weight percent or about 5 to 10% in admixture; titanium dioxide up to 25 weight percent or about 2 to 25% in admixture; and triethanolamine salicylate up to 12 weight percent or about 5 to 12% in admixture.

Typical suitable UV-B type sunscreening actives include benzophenone-3, benzophenone-8, p-methoxycinnamate, substituted para-aminobenzoates, e.g., alkyl (e.g. 2-ethylhexyl) esters of para-methoxycinnamate, octyl methoxycinnamate and octyl para-methoxycinnamate, available from Givaudan Corp., Clifton, N.J. 07104 under the tradename Parsol MCX and usually present in the range of about 2 to 7.5 weight percent, ethylhexyl or octyl salicylate available from Harmann and Riemer, Springfield, N.J., 07081, usually in the range of about 3 to 5 weight percent. The amount of UV-B type sunscreening active should be sufficient to give an SPF of at least 2 to 15.

A suitable UV-A type sunscreen formulation includes such active agents as oxybenzone, usually in the range of about 2 to about 6 weight percent. Sunscreen emulsions containing mixtures of UV-B and UV-A type sunscreen actives may provide an SPF rating of from 2 to 50. These may be formulated to include oils, solvents, emollients, humectants, vitamins, emulsifiers, gels, scenting agents, coloring agents, preservatives, and any other material known to the art for use in sunscreens.

According to the present disclosure, the sunscreen base also contains a color-triggering developer, which serves as a chemical activator or initiator for the conversion of the color precursor into chromophore containing dyes that display colors. When the color precursor is preferably a leuco dye intermediate, the color-triggering developer is a Lewis acid. The most desirable Lewis acids for use as the color-triggering developer are zinc-containing resins. Activated clays and phenolic resins are less desirable because of their relatively slow rate of reactivity. In addition, clays and phenolic resins form color-forming complexes that are undesirably water soluble. This water solubility is problematic because water may remove the color marks from the skin. The color complexes of the marks formed by the use of zinc-containing developers are less water sensitive. Accordingly, the color is less apt to be removed from the skin and tends to be less messy.

Most preferably, an especially desirable color-triggering developer is a zincated carboxylic resin that is dispersed in the sunscreen. For example, the zinc acts as a Lewis acid and causes the rearrangement of the dye molecule, thereby resulting in the development of the desired chromophore. Particularly, the development of the color occurs when the color-triggering developer reacts with the leuco dye intermediate to form a highly conjugated compound thereby resulting in a chromophore of intense color.

In some embodiments, the color-triggering developer is mixed with a sunscreen base formulation to provide a developer concentration of at least about 2% or 3% of the total weight. Increased concentrations provide a stronger color showing, such as a 12% or greater concentration. Levels of color-triggering developer that may be used in the formulations below are suitably from about 2% to 20% by weight of the formulation.

The color-triggering developer is preferably dispersed in water. The sunscreen base is desirably also provided with a void cell former, such as, for example, calcium carbonate. The calcium carbonate is precisely geometrically formed such that it forms a void cell in the sunscreen base. The void cell functions by capillary action. Particularly, the calcium carbonate is designed to hold the developed ink in a cell to prevent ink penetration into and across the skin in order to prevent smearing of the developed ink. If the solvent for the marking composition is non-volatile and not otherwise held in a micelle, the solvent may flow across the surface of the skin and carry the chromophore with it. As a result, the color markings may become fuzzy and indistinct. In the event that a volatile solvent is utilized in the marking composition, the void cell forming species may not be required because the solvent tends to evaporate off on the skin, thereby leaving the dye chromophore locked onto the skin surface. By way of example, and not limitation, the void cell former may be present in a concentration of about 3% to 8% by total weight of the sunscreen formulation.

The calcium carbonate and other solid ingredients are held onto the skin by at least one binder, such as, for example, starch-modified latex. The latex may be cross-linked using a zinc or zirconium salt to enhance the strength of the film. For example, after the sunscreen base dries, cross-linking occurs, especially by application of heat. In order to enhance reactivity between the color-triggering developer and the leuco dye, especially when the marking composition is incorporated into a stick of soft wax or gel, liquid plasticizers (e.g., non-volatile solvents) may be incorporated into the sunscreen base. When the water has evaporated from the sunscreen base and the marking composition is subsequently applied to the skin, the liquid plasticizer can facilitate the ability of the leuco dye to penetrate the sunscreen base more rapidly, thereby resulting in a faster color-formation reaction. Accordingly, the plasticizer increases the reactivity (e.g., by a factor of 10). Examples of plasticizers include, but are not limited to, dibutyl phthalate and citrate ester (e.g., Citroflex A4). In addition, the sunscreen base may also include an oil absorption enhancer, such as, diatomaceous earth.

Alternatively, the sunscreen base may incorporate an encapsulated solvent that desirably ruptures when written upon. The solvent is desirable because it increases the speed of color development and permits the reaction to proceed at a visible rate. For example, employment of an encapsulated solvent is particularly desirable where the marking instrument is in the form of a wax crayon. In this regard, it will be appreciated that wax is a poor electron transfer medium. Consequently, the solvent allows the reaction to proceed at a visible rate. The plasticizers are particularly desirable for inclusion in the encapsulated solvent. Advantageously, the sunscreen product is substantially non-staining to all surfaces other than the skin covered with a film of sunscreen base containing the color-triggering developer.

The color marks formed from the leuco dye color precursor and the Lewis acid color-triggering developer are generally washable in the event that color marks form on unintended surfaces. It is recognized that while formation of color marks on unintended surfaces is generally inhibited, color marks could be formed inadvertently under certain circumstances. For example, laundry detergents contain zwitterions designed to be acidic and basic at the same time to facilitate soil removal. The acids in the laundry detergent can cause color development on fabrics marked with the leuco dyes during washing with most conventional laundry detergents commonly used when washing clothes in a washing machine. In addition, many fabrics, such as cotton, contain natural fatty acids which can trigger color development with leuco dyes, especially when exposed to heat, as is typically found in a clothes dryer. In this regard, heating can accelerate the formation of color. Washability may be assessed as a ΔE value, as described herein below, of 5 or less, and more preferably, 3 or less.

The following examples further illustrate by way of example and should not be construed as in any way limiting its scope. Quantities are in percent by weight of the total composition.

Examples of marking compositions that contain leuco dye intermediates are provided in Examples 1-9, as described in U.S. Pat. No. 6,124,377. These compositions may be used to fill the applicator 104, as shown in FIG. 1, and respectively include color precursor materials to form brown, red, blue, orange, green, black, purple, and yellow colors when eventually reacted with the sunscreen formulation. The marking compositions of Examples 1-9 may be mixed with one another to provide additional colors. The components are added in the order by which they are listed. The components are mixed while heating to approximately 90° C. until the composition is clear. Each marking composition resulted in the formation of a bright, vivid color upon exposure to the color-triggering developer.

Examples 1-8 also illustrate the washability of the formulations of the present invention. In this regard, fabric fugitivity data, stated in terms of ΔE values, can be measured by way of a spectrophotometer. The measured ΔE values correspond to visual observations as follows:

| | |
|---|---|
| ΔE = up to 2.2 | no visible stain |
| ΔE = 2.20-2.90 | trace |
| ΔE = 2.90-4.90 | slight |
| ΔE = 4.90-6.40 | moderate |
| ΔE = over 6.40 | heavy |

In Examples 1-8, the washability (in terms of ΔE) was tested after marking the composition onto a fabric. The fabric was then subjected to washing in a washing machine set on a warm wash and then drying the fabric in a clothes dryer on a hot setting. The washability was then tested and evaluated.

EXAMPLE 1
BROWN

| Component | Weight % |
|---|---|
| dimethyl adipate (solvent) | 62.50 |
| Irganox 1010 (antioxidant) | 10.00 |
| Tridecyl amine (buffer) | 0.25 |
| Trihexyl amine (buffer) | 0.25 |
| Surfynol 104A (surfactant) | 10.00 |
| Dynol 604 (surfactant) | 15.00 |
| Hodogaya Green DCF (color precursor) | 0.50 |
| Hodogaya Orange DCF (color precursor) | 1.50 |
| Total | 100.00 |
| ΔE Wash Value | 2.41 |

EXAMPLE 2
RED

| Component | Weight % |
|---|---|
| dimethyl adipate (solvent) | 62.90 |
| Irganox 1010 (antioxidant) | 10.00 |
| Tridecyl amine (buffer) | 0.25 |
| Trihexyl amine (buffer) | 0.25 |
| Surfynol 104A (surfactant) | 10.00 |
| Dynol 604 (surfactant) | 15.00 |
| Hodogaya Orange DCF (color precursor) | 1.52 |
| COPIKEM .RTM. 7 Grape (color | 0.08 |
| precursor) | |
| Total | 100.00 |
| ΔE Wash Value | 3.41 |

EXAMPLE 3
BLUE

| Component | Weight % |
|---|---|
| dimethyl adipate (solvent) | 62.74 |
| Irganox 1010 (antioxidant) | 10.00 |
| Tridecyl amine (buffer) | 0.25 |
| Trihexyl amine (buffer) | 0.25 |
| Surfynol 104A (surfactant) | 10.00 |
| Dynol 604 (surfactant) | 15.00 |
| Hodogaya Blue NC-1 (color | 1.76 |
| precursor) | |
| Total | 100.00 |
| ΔE Wash Value | 0.82 |

EXAMPLE 4
ORANGE

| Component | Weight % |
|---|---|
| dimethyl adipate (solvent) | 63.00 |
| Irganox 1010 (antioxidant) | 10.00 |
| Tridecyl amine (buffer) | 0.25 |
| Trihexyl amine (buffer) | 0.25 |
| Surfynol 104A (surfactant) | 10.00 |
| Dynol 604 (surfactant) | 15.00 |
| Hodogaya Orange DCF (color | 1.50 |
| precursor) | |
| Total | 100.00 |
| ΔE Wash Value | 2.51 |

EXAMPLE 5
GREEN

| Component | Weight % |
|---|---|
| dimethyl adipate (solvent) | 58.40 |
| Irganox 1010 (antioxidant) | 15.00 |
| Trihexyl amine (buffer) | 0.30 |
| Tridecyl amine (buffer) | 0.30 |
| Surfynol 104A (surfactant) | 10.00 |
| Dynol 604 (surfactant) | 15.00 |
| Hodogaya Green DCF (color | 1.00 |
| precursor) | |
| Total | 100.00 |
| ΔE Wash Value | 3.09 |

EXAMPLE 6
BLACK

| Component | Weight % |
|---|---|
| dimethyl adipate (solvent) | 57.90 |
| Irganox 1010 (antioxidant) | 15.00 |
| trihexyl amine (buffer) | 0.3 |
| tridecyl amine (buffer) | 0.3 |
| Surfynol 104A (surfactant) | 10.00 |
| Dynol 604 (surfactant) | 15.00 |
| Copikem #34 Black | 0.5 |
| (color precursor) | |
| Copikem #4 Black | 0.5 |
| (color precursor) | |
| Hodogaya NC-Black-1 | 0.5 |
| (color precursor) | |
| Total | 100.00 |
| ΔE Wash Value | 2.47 |

EXAMPLE 7
PURPLE

| Component | Weight % |
|---|---|
| dimethyl adipate (solvent) | 58.90 |
| Irganox 1010 (antioxidant) | 15.00 |
| trihexyl amine (buffer) | 0.3 |
| tridecyl amine (buffer) | 0.3 |
| Surfynol 104A (surfactant) | 10.00 |
| Dynol 604 (surfactant) | 15.00 |
| Copikem #7 Grape (color precursor) | 0.5 |
| Total | 100.00 |
| ΔE Wash Value | 2.1 |

EXAMPLE 8
YELLOW

| Component | Weight % |
|---|---|
| dimethyl adipate (solvent) | 57.56 |
| Irganox 1010 (antioxidant) | 15.00 |
| trihexyl amine (buffer) | 0.1 |
| tridecyl amine (buffer) | 0.1 |
| Surfynol 104A (surfactant) | 10.00 |
| Dynol 604 (surfactant) | 15.00 |
| Copikem #37 Yellow (color precursor) | 0.24 |
| Ciba Pergascript Yellow | 2.00 |
| Total | 100.00 |
| ΔE Wash Value | 2.47 |

Example 9 provides an exemplary formulation, as described in U.S. Pat. No. 6,124,377, for the marking composition of the present invention that includes the use of a dye blocker, and particularly a novolac epoxy dye blocker (commercially available as Epon 825 from Shell Chemical). The washability of the formulation was tested on a single layer fabric after use in a washing machine set on a hot wash followed by exposure to heat in a clothes dryer on a hot setting.

EXAMPLE 9

| | Amount |
|---|---|
| Component | (grams) |
| dimethyl adipate (solvent) | 91.61 |
| Epon 825 (dye blocker) | 0.20 |
| Irganox 1010 (antioxidant) | 3.00 |
| trihexyl amine (buffer) | 0.03 |
| tridecyl amine (buffer) | 0.03 |
| Surfynol 104A (surfactant) | 2.00 |
| Dynol 604 (surfactant) | 3.00 |
| Copikem 7 Grape (color precursor) | 0.13 |
| ΔE Wash Value | 2.80 |

A sunscreen formulation material may be prepared to include the following ingredients, using a zincated resin as the color-triggering developer. This sunscreen formulation may be used as the sunscreen covering back 102, as shown in FIG. 1.

EXAMPLE 10
SUNSCREEN FORMULATION

| | | Amount | |
|---|---|---|---|
| Material | Description | (grams) | Wt. Percent |
| Water | city water | 39 | 6.03 |
| Albagloss S | calcium carbonate | 150 | 23.20 |
| Slurry | dispersion | | |
| AMP 95 | Isopropanol Amine | 3 | 0.46 |
| Soribitol | Plasticizer | 12 | 1.66 |
| Celite 281 | diatomaceous Earth | 50 | 7.73 |
| Albagloss S | calcium carbonate | 183 | 26.31 |
| Slurry | slurry | | |
| HRJ 10802 | zincated resin | 166 | 25.68 |
| Pennsize 630 | starch modified SBR latex | 40 | 6.19 |
| Bacote 120 | organic zirconium crosslinker | 0.5 | 0.46 |
| FC 120 | fluorocarbon surfactant | 14.7 | 2.28 |
| Total Weight | | 646.5 | 100.00 |

In making the sunscreen base compositions set forth in Example 10, the city water, Albagloss S slurry (calcium carbonate dispersion), AMP 95, and sorbitol are mixed in a mixer operating at a low speed. Celite 281 is then added while increasing the mixer speed. The mixer speed is held for 10 minutes at a dispersing speed. The mixture is then pumped into a larger mixing tank. The Albagloss S slurry (calcium carbonate slurry), HRJ 10802, Pennsize 630, Bacote 120 and FC 120, are then added and mixed thoroughly for 15 minutes.

The sunscreen base, as prepared above, is mixed with another Lewis acid as the color-triggering developer.

While this invention has been described with an emphasis upon certain preferred embodiments, it will be apparent to those of ordinary skills in the art that variations in the preferred compositions and systems may be used and that it is intended that the invention may be practiced otherwise than as specifically described herein. Accordingly, this invention includes all modifications encompassed within the spirit and scope of the invention as defined by the following claims.

What is claimed is:

1. A sunscreen system for protection of human skin, the improvement comprising:
a sunscreen formulation including a color-triggering developer; and
a composition including at least one color precursor capable of reacting with the color-triggering developer to provide a coloration of sufficient intensity to function as an indicator that the sunscreen is present when the sunscreen is applied to the human skin.

2. The system of claim 1, further including means for applying the composition to a selected area of the human skin.

3. The system of claim 2, wherein the means for applying comprises a stamp.

4. The system of claim 3, further including a stamp pad or reservoir for dispensing the composition to the stamp.

5. The system of claim 1, wherein the composition contains at least one antioxidant selected from the group consisting of 3,5-bis (1,1-dimethylethyl)-4-hydroxy benzenepropanoic acid,2,2-bis[[3-[3.5-bis(1,1-dimethylethyl)-4-hydroxyphenyl]-1-oxopropoxy]methyl]1,3-propanediyl ester; Vitamin E; Tinuvins; butylated hydroxytoluene; butylated hydroxyanisole; and any combination thereof, the antioxidant being present in a concentration of from about 1% to about 30% by weight of the composition.

6. The system of claim 1, wherein the sunscreen formulation includes a buffer selected from the group consisting of non-water-soluble tertiary amines, and combinations thereof.

7. The system of claim 6, wherein the buffer comprises the non-water-soluble tertiary amine selected from the group consisting of tributyl amine, trihexyl amine, tridodecyl amine, tridecyl amine, trioctyl amine, and combinations thereof, the non-water-soluble tertiary amine is present in a concentration of from about 0.0002% to about 1.5% by weight of the composition.

8. The system of claim 1, wherein the color precursor includes a leuco dye intermediate in a concentration ranging from about 0.1% to about 10% by weight of the composition.

9. The system of claim 8, wherein the leuco dye intermediate is not water soluble.

10. The system of claim 8, wherein the leuco dye intermediate is selected from the group consisting of diarylphthalide dyes, fluoran dyes, indolyphthalide dyes, acylluecoazine dyes, leucoauramine dyes, spiropyrane dyes, rhodaminelactam dyes, triarylmethane dyes, and chromene dyes, and combinations thereof.

11. The system of claim 8, wherein the leuco dye intermediate is selected from the group consisting of Spiro(12H-benzo(a)xanthene-12,1′(3′H)isobenzofuran-3′-one,9-(diethylamino),3-[Butyl-2-methylindol-3-yl]-3-(1-octyl-2-methylindol-3-yl)-1(3H)isobenzofuranone, 2-′phenylamino-3′-methyl-6′-(dibutylamino)spiro[isobenzofuran-1(3H),9′-(9H)-xanthen]-3-one, substituted Phthalide, 2′-Di(phenylmethyl)amino-6′-(diethylamino)spiro(isobenzofuran-1(3H),9′(9H) xanthen)-3-one.

12. The system of claim 1, wherein the composition comprises at least one surfactant having an hydrophilic-lipophilic balance of about 6 or less.

13. The system of claim 1, wherein the composition comprises at least one surfactant forming a surfactant blend, wherein the surfactant blend has a hydrophilic-lipophilic balance ranging from about 3 to about 6.

14. The system of claim 1, wherein the composition comprises at least one surfactant selected from the group consisting of acetylinic diol based surfactants, ethoxylated acetylinic diols, dibasic ester microemulsion concentrates including water, and combinations thereof.

15. The system of claim 1, wherein the color-triggering developer includes a Lewis acid material.

16. The system of claim 15, wherein the Lewis acid material is selected from the group consisting of activated clays, phenolic resins, zinc containing resins, and combinations thereof.

17. The system of claim 15, wherein the Lewis acid material includes a zinc-containing resin.

18. The system of claim 17, wherein the color-triggering developer comprises a zincated carboxylic resin.

19. The system of claim 1, wherein the weight of the color-triggering developer ranges from 4% to 20% by weight of the sunscreen formulation.

20. The system of claim 1, wherein the weight of the color-triggering developer is less than about 12% by weight of the sunscreen formulation.

21. The system of claim 1, wherein the sunscreen formulation further comprises a void cell former.

22. The system of claim 21, wherein the void cell former comprises calcium carbonate.

23. The system of claim 1, further comprising an applicator for applying the composition to the sunscreen formulation when the sunscreen formulation is applied to the human skin, the applicator selected from the group consisting of markers, brushes, paintbrushes, pencils, pens, stamps, stamp pads, spray bottles, fingers, and crayons.

24. The system of claim 1, wherein the composition includes at least one surfactant, at least one antioxidant, and at least one buffer.

25. The system of claim 24, wherein
at least one antioxidant comprises at least one of 3,5-bis (1,1-dimethylethyl)-4-hydroxy benzenepropanoic acid, and 2,2-bis[[3-[3.5-bis(1,1-dimethylethyl)-4-hydroxyphenyl]-1-oxopropoxy]methyl]1,3-propanediyl ester;
at least one buffer is selected form the group consisting of trihexyl amine, tridecyl amine, and combinations thereof; and
at least one surfactant is selected from the group consisting of acetylinic diol based surfactants, ethoxylated acetylinic diol based surfactants, and combinations thereof.

26. The system of claim 24, wherein
at least one antioxidant comprises at least one of 3,5-bis (1,1-dimethylethyl)-4-hydroxy benzenepropanoic acid, and 2,2-bis[[3-[3.5-bis(1,1-dimethylethyl)-4-hydroxyphenyl]-1-oxopropoxy]methyl]1,3-propanediyl ester;
at least one buffer is selected from the group consisting of trihexyl amine, tridecyl amine, and a combinations thereof; and
at least one surfactant is selected from the group consisting of acetylinic diol based surfactants, ethoxylated acetylinic diol based surfactants, and combinations thereof.

27. A system comprising:
a sunscreen formulation;
a color-triggering developer;
a composition comprising at least one color precursor and at least one additive selected from the group consisting of surfactants, antioxidants, buffers, and any combination thereof; and
means for applying said composition to the mixture of sunscreen base and the color-triggering developer to form at least one color mark.

28. The system of claim 27 wherein the sunscreen base and the color-triggering developer are in a mixed state combining the sunscreen base and the color-triggering developer.

29. The system of claim 27 wherein the sunscreen base and the color-triggering developer are separate from one another.

30. A method of protecting human skin by the application of sunscreen, comprising:
applying to human skin a sunscreen formulation that contains a color-triggering developer; and thereafter
applying to the sunscreen formulation a composition that contains a color precursor for reaction with the color-triggering developer;
the reaction providing a colored indicator that the sunscreen is present on the human skin.

31. The method of claim 30, further comprising monitoring the colored indicator for signs of visible fading.

32. The method of claim 31, further comprising reapplying the sunscreen formulation when visible fading is perceived.

33. The method of claim 30, wherein the step of applying includes applying the composition to a selected portion of the sunscreen formulation on the human skin.

34. The method of claim 30, wherein the selected portion includes a pattern or design.

35. The method of claim 30, wherein the step of applying includes stamping.
